SEQUENCE LISTING (4 sequences)

SEQ ID NO: 1 - protein, 14 aa, Homo sapiens
CVVSDRGSTLGRLY

SEQ ID NO: 2 - protein, 14 aa, Macaca fascicularis
CVVSDRGSTLGKLY

SEQ ID NO: 3 - protein, 449 aa, Artificial sequence
  NKTT320 Heavy chain sequence (Translation of VH4-G4)
EVQLVESGGGLVQPGGSLRLSCVASGFTFSNYWMNWVRQAPGKGLEWVAEIRLKSNNYATHYAESVKGRFTISRDDSKNTVYLQMNSLRAEDTAVYYCTRNGNYVDYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 214 aa, Artificial sequence
  NKTT320 Light chain sequence (Translation of VK4-CK)
DIQMTQSPSSLSASVGDRVTITCKASQDVSTAVAWYQQKPGQAPRLLIYWASTRHTGVPSRFSGSGSGTDFTLTISSLQPEDFALYYCQQHYSTPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2016/0022777 A1
(43) Pub. Date: Jan. 28, 2016

(54) COMBINATION THERAPY WITH IL-12

(71) Applicant: NKT Therapeutics Inc., Waltham, MA (US)

(72) Inventors: Robert Schaub, Waltham, MA (US); Hans Albert Felix Scheuplein, Arlington, MA (US)

(21) Appl. No.: 14/810,587

(22) Filed: Jul. 28, 2015

Related U.S. Application Data
(60) Provisional application No. 62/029,908, filed on Jul. 28, 2014.

(51) Int. Cl. A61K 38/20; A61J 1/05; A61K 39/395

(52) U.S. Cl. CPC A61K 38/208; A61K 39/39541; A61J 1/05; A61K 2039/505

(57) ABSTRACT

Pharmaceutical compositions and treatments involving iNKT cell activation are provided.

### RELATED APPLICATIONS

This application claims the benefit under 35 U.S.C. §119(e) of U.S. provisional application Ser. No. 62/029,908, filed Jul. 28, 2014, the content of which is incorporated by reference herein in its entirety.

### BACKGROUND OF THE INVENTION

Modulating the immune system has been pursued as a desirable approach to treat a variety of diseases and disorders, including, but not limited to, autoimmune disease, infection, allergy, inflammatory conditions, spontaneous abortion, pregnancy, graft versus host disease and cancer. T cells have been a target of such modulation. T cells are lymphocytes that participate in multiple immune system functions. Subsets of T cells such as helper T cells, cytotoxic T cells and suppresser T cells, mediate different immunologic functions. Natural killer T (NKT) cells are a subset of T lymphocytes that share surface markers and functional characteristics with both conventional T cells and natural killer (NK) cells. Unlike T cells, they recognize glycolipid antigens rather than peptide antigens.

NKT cells can be divided into three subsets: Type 1 which express an invariant T cell receptor and are CD1d-restricted (iNKT), Type 2 (NKT) which express varied T cell receptors, but are CD1d-restricted, and Type 3 which do not express the invariant T cell receptor and are not CD1d-restricted (NKT-like). Type 1 iNKT cells express a uniquely rearranged, highly conserved, semi-invariant TCR-α chain (Vα24-Jα18 in humans and Vα14-Jα18 in mice), which preferentially pairs with specific TCR-β chains (Vβ11 in humans or Vβ8.2, Vβ7 and Vβ2 in mice). They are highly conserved throughout animal phylogeny. This is in contrast to most T cell subpopulations, which have diverse sequences for their T Cell Receptors (TCRs). The invariant TCR of iNKT cells recognizes glycolipid antigens presented on the MHC-I-like protein CD1d on the surface of antigen presenting cells. A hallmark of iNKT cells is their capacity to rapidly produce a mixture of cytokines, including IFNγ and IL-4 , which are signature cytokines otherwise produced by T helper type I (Th1) and Th2 cells, respectively. Invariant NKT cells are sometimes referred to as “Classical NKT Cells”.

A unique feature of iNKT cells is that they recognize and are activated by the marine sponge-derived glycolipid, α-Galactosyl-Ceramide (α-GalCer), presented on CD1d. This has been utilized, for example, to monitor iNKT cells by flow cytometry, by using α-GalCer-loaded CD1d tetramers. The mouse monoclonal antibody 6B11, which binds to the invariant loop of the human-iTCR, has also been used to monitor human and NHP iNKT cells. The pathways associated with iNKT cell activation by α-GalCer also have been studied.

iNKT cells develop in the thymus, similar to other T cells. Studies in mice show that iNKT cells, unlike conventional T cells, acquired a memory phenotype during their natural development by recognizing hitherto unknown, endogenous antigens presented on CD1d molecules, and without requiring prior exposure to foreign or pathogenic antigens. Due to their memory phenotype, they can be rapidly activated and expand within the peripheral immune compartment in response to exposure to foreign or endogenous glycolipid antigens presented by antigen-presenting cells (APCs).

iNKT cells share characteristics of both the innate and adaptive arms of the immune system and thus play a unique role by modulating T and B cell responses as well as innate immunity. iNKT cells are rapid-onset which is a feature of the innate immune system. They also display features of the adaptive immune system because they share properties of other T cells such as antigen specific responses. As such, they serve as a bridge between the two systems where they can play both pro-inflammatory and immuno-regulatory roles either to enhance or attenuate developing immune responses, respectively.

The properties of iNKT cells has prompted investigations into the manipulation of iNKT cell function as a treatment for disease. Numerous studies have shown that iNKT cells can regulate the balance between Th1 and Th2 responses. These cells are postulated to play a role in the response to pathogens, in immune surveillance in cancer, and in the regulation of autoimmunity. For most of these conditions, the iNKT cell defect has only been partially characterized and in some cases has been disputed by contradictory studies. Human studies, in particular, are constrained by two important limitations. First, most human studies have used suboptimal methods for the identification of iNKT cells. Second, most human studies are qualitative only, and little human data exists respecting the functional consequences of modulation of iNKT cell numbers, ratios, or function.

Studies to date have used an indirect approach to iNKT cell stimulation via the presentation of the activating glycolipid ligand alpha galactosylceramide or other stimulatory glycolipids by dendritic cells or other antigen presenting cells (APCs) that express the MHC class I like molecule CD1d. What is not known is if direct activation of iNKT cells by use of a direct iNKT binding antibody without the presentation of a glycolipid or activating cytokines via APC/iNKT cell binding can have a similar effect.

The PCT published application WO 2013/063395 discloses for the first time humanized antibodies that bind human iNKT cells, including antibodies that can activate iNKT cells in vivo and antibodies that can deplete iNKT cells in vivo. This application provides for the first time the opportunity to test such antibodies in human systems as therapeutics for the treatment of disease.

Certain cancers have been reported to interfere with iNKT cell activation. Recently it was reported that iNKT activation could be restored in a mouse model with such a cancer. The investigators showed an improved cytokine response by iNKT cells when α-GalCer was combined with IL-12 in a mouse which had received cancer cells known to suppress iNKT activation. Treatment of cancer was not shown.

### SUMMARY OF THE INVENTION

It has been discovered that combination treatment with both an antibody that activates iNKT cells and IL-12 is surprisingly effective and synergistic in treating cancer and preventing cancer metastasis. It is also believed that these findings extend to infectious disease. The treatment is effective even without separate administration of an isolated antigen, although the treatment does not rule out separate treatment with an isolated antigen.

It also has been demonstrated that direct activation of iNKT cells can adequately stimulate iNKT cells to provide both prophylactic and therapeutic anti-tumor activity when administered. These results suggest a new approach to the activation of iNKT cells for the treatment of cancer.

According to one aspect of the invention, a method is provided for treating a human subject having a cancer or an infection. The method comprises administering to the human subject an effective amount of (a) an isolated humanized antibody that selectively binds and activates iNKT cells, and (b) rhIL-12, wherein the isolated humanized antibody and the rhIL-12 are administered in amounts effective to treat the cancer or the infection. In some embodiments, the treatment is without concurrent administration of an antigen containing vaccine. In some embodiments, the treatment further comprises administering an antigen containing vaccine. In embodiments the treatment can be without concurrent treatment with α-GalCer.

The cancer can be, for example, melanoma, squamous cell carcinoma, basal cell carcinoma, breast cancer, head and neck carcinoma, thyroid carcinoma, soft tissue sarcoma, bone sarcoma, testicular cancer, prostatic cancer, ovarian cancer, bladder cancer, skin cancer, brain cancer, angiosarcoma, hemangiosarcoma, mast cell tumor, primary hepatic cancer, lung cancer, pancreatic cancer, gastrointestinal cancer, renal cell carcinoma, hematopoietic neoplasia, or a metastatic cancer thereof. In some embodiments, the cancer is melanoma. In some embodiments, the cancer is renal cell carcinoma.

In embodiments, the dose of rhIL-12 can be reduced substantially below the reported ‘optimal’ dosing of 500 ng/kg. In particular, according to the invention, the daily dose of rhIL-12 does not exceed 250 ng/kg per day, does not exceed 200 ng/kg per day, does not exceed 150 ng/kg per day, does not exceed 100 ng/kg per day, does not exceed 75 ng/kg per day, does not exceed 50 ng/kg per day, and even does not exceed 25 ng/kg per day.

In some embodiments, the rhIL-12 is administered in a single injection between one and three weeks prior to daily consecutive dosing, and then is administered in daily consecutive dosing for at least 5 days and not more than 10 days.

In embodiments, the isolated humanized antibody and the rhIL-12 are not administered on the same day. In embodiments, the isolated humanized antibody and the rhIL-12 are administered on the same day. In embodiments the isolated humanized antibody is administered in a first dose on a single day and the rhIL-12 is administered on multiple days.

In embodiments, the subject is not receiving concurrent therapy with α-GalCer.

In embodiments, the isolated humanized antibody that selectively binds and activates iNKT cells is NKTT320.

According to another aspect of the invention, a kit is provided. The kit includes a package containing (a) an isolated humanized antibody that selectively binds and activates iNKT cells, and (b) rhIL-12. In embodiments, the isolated humanized antibody that selectively binds and activates iNKT cells is NKTT320. In embodiments, the isolated humanized antibody that selectively binds and activates iNKT cells and the rhIL-12 are in separate containers within the package. In some embodiments, the kit does not include an antigen containing vaccine. In some embodiments, the kit includes an antigen containing vaccine.

In any of the embodiments above, the isolated humanized antibody that selectively binds and activates iNKT cells can be any of the humanized antibodies that selectively bind and activate iNKT cells described in greater detail below. In any of the embodiments above, the isolated humanized antibody that selectively binds and activates iNKT cells can be NKTT320.

### BRIEF DESCRIPTION OF THE DRAWINGS

FIG. 1 shows the cytokine profile induced by NKT14m and by α-GalCer in human iNKT cells.

FIG. 2 shows the chemokine profile induced by NKT14m and by α-GalCer in human iNKT cells.

FIG. 3 shows the effects on several parameters after six weeks dosing with α-GalCer versus NKT14m.

FIG. 4 is a graph showing the effects on melanoma metastases in the lungs of mice receiving tumor cells, and then treated with control, rhIL-12 alone, NKT14m alone, and rhIL-12 plus NKT14m.

### DETAILED DESCRIPTION

The following detailed description is made by way of illustration of certain aspects of the invention. It is to be understood that other aspects are contemplated and may be made without departing from the scope or spirit of the present disclosure. The following detailed description, therefore, is not to be taken in a limiting sense. Scientific and technical terms used herein have meanings commonly used in the art unless otherwise specified. The definitions provided herein are to facilitate understanding of certain terms used frequently herein and are not meant to limit the scope of the present disclosure. The singular forms “a”, “an”, and “the” encompass the plural, unless the content clearly dictates otherwise. The term “or” is generally employed in its sense including “and/or” unless the content clearly dictates otherwise.

Antibody that activates iNKT cells. The invention involves use of an antibody that activates iNKT cells. Such an “Activating Antibody” is one that, when it binds to an iNKT cell in vivo, results in stimulating the iNKT cell to produce interferon gamma, IL4, IL10, or IL13 versus blocking the activity of or depleting the iNKT cell. Blocking the activity means that when the antibody binds to an iNKT cell in vivo, it results in preventing or lessening the ability of the iNKT cell to produce interferon gamma, IL4, IL10, and/or IL13. Such blocking antibodies include antibodies that result in depletion of iNKT cells when the antibody binds to an iNKT cell in vivo. Activating Antibodies typically have an Fc portion that does not bind FcγRI and C1q. In one embodiment, the Fc portion of the Activating Antibody does not bind FcγRI, C1q, or FcγRIII. Antibodies with such functionality, in general, are known. There are native antibodies, such as antibodies with an IgG4 Fc region. There also are antibodies with Fc portions genetically or chemically altered to eliminate the Antibody dependent cell cytotoxicity (ADCC) and/or complement dependent cytotoxicity (CDC) functionality.

IgG4 has been used as an Activating Antibody, as it has a low affinity for FcgammaRI. IgG4 can be modified to decrease its Fc effector functions, making it even more suitable as an Activating Antibody. For example, an Fc region modified by two single residue substitutions is described in “Elimination of Fc Receptor-dependent Effector Function of a Modified IgG Monoclonal Antibody to Human CD4”, Truneh et al., The Journal of Immunology, 1925-1933, 2000. Changes to the glycosylation of the Fc region also have been made to improve antibody based therapeutics. See for example, Glycosylation as a strategy to improve antibody-based therapeutics”, Jefferis, R., Nature Reviews, Vol 8, March 2009, 226-234. All such activating modifications as described above are within the scope of the present invention.

In an embodiment, the antibody binds selectively the epitope defined by SEQ ID No. 1. In other embodiments, the antibody binds selectively the epitope defined by both SEQ ID Nos. 1 and 2.

NKTT320 is a humanized mAb which specifically recognizes human iTCR. It is a modified IgG4 mAb with two amino acid changes introduced in the hinge region, one designed to stabilize the IgG4 heavy chain dimer formation, and the 2nd to reduce residual FcγR binding capacity. NKTT320 comprises a light chain containing SEQ ID NO:4 and a heavy chain containing SEQ ID NO: 3. NKTT320 and numerous other iNKT Activating Antibodies are described in U.S. patent application publication number 2013/0136735, the entire disclosure of which is incorporated herein by reference.

Recombinant Human Interleukin 12 (rhIL-12). Interleukin 12 (IL-12) is a disulphide-bonded heterodimer consisting of a 35 kDa alpha subunit and a 40 kDa beta subunit. It is involved in the stimulation and maintenance of Th1 cellular immune responses, including the normal host defense against various intracellular pathogens. A recombinant form of the endogenous heterodimeric cytokine interleukin-12 is available from several commercial sources. Recombinant human interleukin-12 has been used in clinical trials, alone and with other agents, in the attempted treatment of cancer and infectious disease.

Recombinant human IL-12 is extremely toxic, and several patients died in one clinical study involving rhIL-12. The dosing in that study was 500 ng/kg. It was believed that 500 ng/kg was optimal dosing, but that the patients died because the patients were not first ‘tolerized’ to the treatment by providing a first dose one—three weeks in advance of more frequent dosing.

rhIL-12 is typically dosed either iv or subcutaneous. It is believed, based on the prior art, that it may be desirable to give a first dose 1-3 weeks prior to starting more frequent dosing, such as a first dose on day one and then after 1-3 weeks, dosing every day, every other day, every third day, or every fourth day, and the like. The initial ‘tolerizing’ dose may not be necessary according to the invention because the daily dose is dramatically reduced according to the invention. Dosing can be spaced apart in schedules, such as every day for 5-10 days, and then a rest period, and then another stretch of every day for 5-10 days, then a rest period, and then another stretch of every day for 5-10 days, etc.

According to the present invention, when combining rhIL-12 with the anti-iNKT antibody treatment, the dose of rhIL-12 can be reduced substantially below the reported ‘optimal’ dosing of 500 ng/kg. In particular, according to the invention, the daily dose of rhIL-12 does not exceed 250 ng/kg per day, does not exceed 200 ng/kg per day, does not exceed 150 ng/kg per day, does not exceed 100 ng/kg per day, does not exceed 75 ng/kg per day, does not exceed 50 ng/kg per day, and even does not exceed 25 ng/kg per day.

Isolated. The antibodies and other therapeutic molecules used herein are isolated. Isolated means, in the context of an antibody or other biologic, the antibody or other biologic has been removed from its natural milieu or has been altered from its natural state. As such, isolated does not necessarily reflect the extent to which the molecule has been removed from its natural milieu or has been altered from its natural state. However, it will be understood that an antibody or other biologic that has been purified to some degree and to an extent to which it can be used for its intended therapeutic purpose is “isolated”.

Antibody. The methods herein employ antibodies. The term antibody is used in the broadest sense and specifically includes, for example, single monoclonal antibodies, antibody compositions with polyepitopic specificity, single chain antibodies, and antigen-binding fragments of antibodies. An antibody may include an immunoglobulin constant domain from any immunoglobulin, such as IgG1, IgG2, IgG3, or IgG4 subtypes, IgA (including IgA1 and IgA2), IgE, IgD or IgM.

An antigen-binding fragment means a portion of an intact antibody that binds antigen. Examples of antibody fragments include Fab, Fab′, F (ab′).sub.2, and Fv fragments; diabodies; linear antibodies (Zapata et al., Protein Eng. 8 (10): 1057-1062 [1995]); and single-chain antibody molecules. Fv is the minimum antibody fragment containing a complete antigen-recognition binding site. This region consists of a dimer of one heavy- and one light-chain variable domain in tight, non-covalent association. In this configuration the three CDRs of each variable domain interact to define an antigen-binding site on the surface of the V.sub.H-V.sub.L dimer. Collectively, the six CDRs confer antigen-binding specificity to the antibody. The Fab fragment also contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. F(ab′).sub.2 antibody fragments originally were produced as pairs of Fab′ fragments which have hinge cysteines between them. Other chemical couplings of antibody fragments are also known. In important embodiments, the antibody is a full length antibody (i.e., contains an Fc region, which can be IgG4 for example).

The antibodies used herein are humanized. Humanized forms of non-human (e.g., murine) antibodies then are chimeric immunoglobulins (including full length immunoglobulins), immunoglobulin chains or fragments thereof (such as Fv, Fab, Fab′, F(ab′)₂, scFv or other antigen-binding subsequences of antibodies) which contain minimal sequence derived from the non-human immunoglobulin. Humanized antibodies typically include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues that are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al., Nature, 321:522-525 (1986); Riechmann et al., Nature, 332:323-327 (1988); and Presta, Curr. Op. Struct. Biol., 2:593-596 (1992)).

A composite antibody is an antibody that contains sequence segments from different antibodies. Humanized antibodies can be formed of a composite of overlapping human sequences, with one segment of the CDR found in one human sequence and another segment of the same CDR found in another human sequence, each of the two sequences having common sequences at an overlapping region where the segments meet. In an embodiment, the composite human sequence is free of known T cell epitopes. In embodiments, the composite human sequence does not elicit an immune response in humans. In any of the embodiments, the subject can be human and the antibody can be a humanized antibody. In any of the embodiments, the antibody can be a composite antibody. In any of the embodiments, the subject can be human and the antibody can be a fully human antibody. A fully human antibody is an antibody consisting only of human amino acid sequences.

Further details respecting antibodies and general methods of making antibodies can be found in U.S. patent application publication number 2013/0136735, the entire disclosure of which is incorporated herein by reference.

The antibodies bind selectively their targets, such as iNKT cells, PD-1, PD-L1, PD-L2, CTLA-4 and CTLA-4 ligands. An antibody that binds selectively its target cells means it has the ability to be used in vitro or in vivo to bind to and distinguish such target bearing tissue from other tissue types of the species, including other closely related cell types (e.g., in the case iNKT cells, distinguishing iNKT cells from other types of NKT cells, other lymphocyte types, and all other tissue types) under the conditions in which the antibody is used, such as under physiologic conditions. In an embodiment, the antibody binds selectively human iNKT cells. In an embodiment, the antibody binds to the CDR3 loop of iNKT cells.

Established cancer. The therapies described herein include treatment of an existing or ‘established’ cancer, that is, one that exists and is detectable in the subject.

Infection. The invention is expected to be useful in treating infections, including established and even chronic infections, including viral infection, bacterial infection, fungal infection and parasitic infection. Thus, the disclosed combination therapy is useful to treat viral infections including, but are not limited to, immunodeficiency (e.g., HIV), papilloma (e.g., HPV), herpes (e.g., HSV), encephalitis, influenza (e.g., human influenza virus A), hepatitis (e.g. HCV, HBV), and common cold (e.g., human rhinovirus).

Non-viral infections treatable by the invention include, but are not limited to, infections cause by microoganisms including, but not limited to, Actinomyces, Anabaena, Bacillus, Bacteroides, Bdellovibrio, Bordetella, Borrelia, Campylobacter, Caulobacter, Chlamydia, Chlorobium, Chromatium, Clostridium, Corynebacterium, Cytophaga, Deinococcus, Escherichia, Francisella, Halobacterium, Heliobacter, Haemophilus, Hemophilus influenza type B (HIB), Hyphomicrobium, Legionella, Leptspirosis, Listeria, Meningococcus A, B and C, Methanobacterium, Micrococcus, Myobacterium, Mycoplasma, Myxococcus, Neisseria, Nitrobacter, Oscillatoria, Prochloron, Proteus, Pseudomonas, Phodospirillum, Rickettsia, Salmonella, Shigella, Spirillum, Spirochaeta, Staphylococcus, Streptococcus, Streptomyces, Sulfolobus, Thermoplasma, Thiobacillus, Treponema, Vibrio, Yersinia, Cryptococcus neoformans, Histoplasma sp. (such as Histoplasma capsulatum), Candida albicans, Candida tropicalis, Nocardia asteroides, Rickettsia ricketsii, Rickettsia typhi, Leishmania, Mycoplasma pneumoniae, Chlamydial psittaci, Chlamydial trachomatis, Plasmodium sp. (such as Plasmodium falciparum), Trypanosoma brucei, Entamoeba histolytica, Toxoplasma gondii, Trichomonas vaginalis and Schistosoma mansoni.

The treatment according to the invention may also be with or without concurrent treatment with an antigen containing vaccine. Suitable antigens used in vaccines are well known in the art and will not be listed here.

Subject. “Subject” means a mammal, such as a human, a nonhuman primate, a dog, a cat, a sheep, a horse, a cow, a pig or a goat. In an important embodiment, the mammal is a human.

Treatment. “Treat”, “treating” and “treatment” encompass an action that occurs while a subject is suffering from a condition which reduces the severity of the condition (or a symptom associated with the condition) or retards or slows the progression of the condition (or a symptom associated with the condition). This is therapeutic treatment. “Treat”, “treating” and “treatment” also encompasses an action that occurs before a subject begins to suffer from the condition (or a symptom associated with the condition) and which inhibits the onset of or reduces the severity of the condition (or a symptom associated with the condition). This is prophylactic treatment.

Subjects are treated with effective amounts of the solutions of the invention. An “effective amount” of a compound generally refers to an amount sufficient to elicit the desired biological response, i.e., treat the condition. As will be appreciated by those of ordinary skill in this art, the effective amount of a compound described herein may vary depending on such factors as the condition being treated, the mode of administration, and the age and health of the subject.

For therapeutic treatment, an effective amount is an amount sufficient to provide a therapeutic benefit in the treatment of a condition or to reduce or eliminate one or more symptoms associated with the condition. This may encompass an amount that improves overall therapy, reduces or avoids symptoms or causes of the condition, or enhances the therapeutic efficacy of another therapeutic agent.

For prophylactic treatment, an effective amount is an amount sufficient to prevent, delay the onset of, or reduce the severity of a condition, or one or more symptoms associated with the condition, or prevent its recurrence. This may encompass an amount that improves overall prophylaxis or enhances the prophylactic efficacy of another prophylactic agent.

In general, effective amounts are administered to enhance an immune response in the subject. In connection with a specific disease or condition, “enhance an immune response” means to halt the development of, inhibit the progression of, reverse the development of, or otherwise reduce or ameliorate one or more symptoms of the disease or condition, for example, one or more symptoms of cancer or infectious disease.

Pharmaceutical compositions. Humanized antibodies, biologics and other molecules can be administered for the treatment of various disorders in the form of pharmaceutical compositions. Such compositions include the therapeutic(s) and one or more other pharmaceutically acceptable components. See Remington's Pharmaceutical Science (15th ed., Mack Publishing Company, Easton, Pa. (1980)). The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to adversely affect the biological activity of the antibody. Examples of such diluents are distilled water, physiological phosphate-buffered saline, Ringer's solutions, dextrose solution, and Hank's solution. In addition, the pharmaceutical composition or formulation may also include other carriers or nontoxic, nontherapeutic, nonimmunogenic stabilizers and the like.

Pharmaceutical compositions can also include large, slowly metabolized macromolecules such as proteins, polysaccharides such as chitosan, polylactic acids, polyglycolic acids and copolymers (such as latex functionalized SEPHAROSE™ (GE Healthcare Bio-Sciences Ltd.), agarose, cellulose, and the like), polymeric amino acids, amino acid copolymers, and lipid aggregates (such as oil droplets or liposomes).

Pharmaceutical compositions may be injectable compositions. Injectable compositions include solutions, suspensions, dispersions, and the like. Injectable solutions, suspensions, dispersions, and the like may be formulated according to techniques well-known in the art (see, for example, Remington's Pharmaceutical Sciences, Chapter 43, 14th Ed., Mack Publishing Co., Easton, Pa.), using suitable dispersing or wetting and suspending agents, such as sterile oils, including synthetic mono- or diglycerides, and fatty acids, including oleic acid.

Injectable compositions that include an antibody or other biologic useful in the invention may be prepared in water, saline, isotonic saline, phosphate-buffered saline, citrate-buffered saline, and the like and may optionally mixed with a nontoxic surfactant. Dispersions may also be prepared in glycerol, liquid polyethylene, glycols, DNA, vegetable oils, triacetin, and the like and mixtures thereof. Under ordinary conditions of storage and use, these preparations may contain a preservative to prevent the growth of microorganisms. Pharmaceutical dosage forms suitable for injection or infusion include sterile, aqueous solutions or dispersions or sterile powders comprising an active ingredient which powders are adapted for the extemporaneous preparation of sterile injectable or infusible solutions or dispersions. Preferably, the ultimate dosage form is a sterile fluid and stable under the conditions of manufacture and storage. A liquid carrier or vehicle of the solution, suspension or dispersion may be a solvent or liquid dispersion medium comprising, for example, water, ethanol, a polyol such as glycerol, propylene glycol, or liquid polyethylene glycols and the like, vegetable oils, nontoxic glyceryl esters, and suitable mixtures thereof. Proper fluidity of solutions, suspensions or dispersions may be maintained, for example, by the formation of liposomes, by the maintenance of the desired particle size, in the case of dispersion, or by the use of nontoxic surfactants. The prevention of the action of microorganisms can be accomplished by various antibacterial and antifungal agents, for example, parabens, chlorobutanol, phenol, sorbic acid, thimerosal, and the like. Isotonic agents such as sugars, buffers, or sodium chloride may be included. Prolonged absorption of the injectable compositions can be brought about by the inclusion in the composition of agents delaying absorption—for example, aluminum monostearate hydrogels and gelatin. Solubility enhancers may be added.

Sterile injectable compositions may be prepared by incorporating the therapeutic in the desired amount in the appropriate solvent with various other ingredients, e.g. as enumerated above, and followed by sterilization, as desired, by, for example filter sterilization. In the case of sterile powders for the preparation of sterile injectable solutions, methods of preparation include vacuum drying and freeze-drying techniques, which yield a powder of the active ingredient plus any additional desired ingredient present in a previously sterile-filtered solution. Any suitable sterilization process may be employed, such as filter sterilization, e.g. 0.22 micron filter or nanofiltration, gamma or electron beam sterilization, or pulsed white light. Other suitable sterilization processes include UtiSter (Pegasus Biologics, Irvine Calif.) and those described in, e.g., U.S. Pat. No. 6,946,098 and U.S. Pat. No. 5,730,933.

In various embodiments, the final solution typically is adjusted to have a pH between about 4 and about 9, between about 5 and about 7, between about 5.5 and about 6.5, or about 6. The pH of the composition may be adjusted with a pharmacologically acceptable acid, base or buffer. Hydrochloric acid is an example of a suitable acid, and sodium hydroxide is an example of a suitable base. The hydrochloric acid or sodium hydroxide may be in any suitable form, such as a 1N solution

A resultant injectable solution preferably contains an amount of one or more therapeutics effective to treat a disease. In various embodiments, a therapeutic such as an antibody is present in an injectable composition at a concentration between about 0.0001 mg/ml and about 50 mg/ml. In various embodiments, an antibody is present in an injectable composition at a concentration between about 0.01 mg/mL and about 10 mg/mL.

Biologics such as antibodies also may be administered via other modes of administration known in the art. Such modes of administration include inhalation, ingestion and topical application. Oral administration is also possible for therapeutics, although this form of administration is more challenging for certain biologics such as antibodies.

### EXAMPLE 1

A murine specific antibody (NKT14m) was created that can bind specifically to and activate murine iNKT cells. The specificity of NKT14m—that is, its ability to distinguish iNKT cells from other cells including other NKT cells, was demonstrated and shown to be similar to the specificity in binding observed for NKTT320 which binds specifically human iNKT cells (but not murine iNKT cells).

### EXAMPLE 2

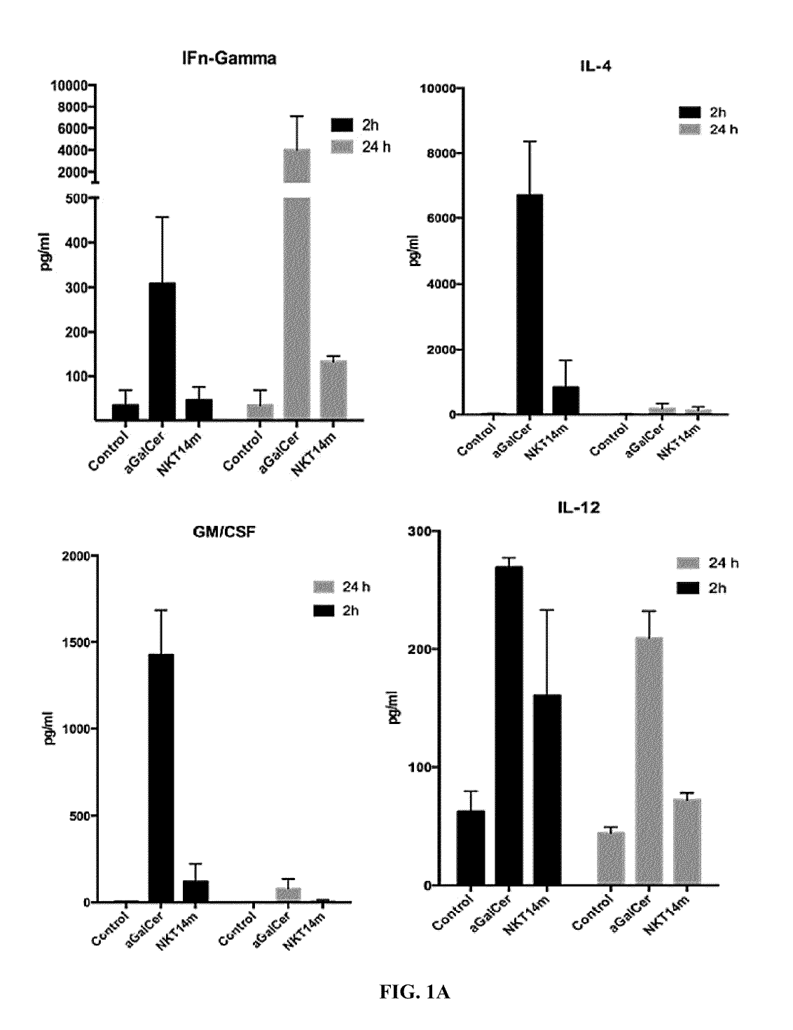

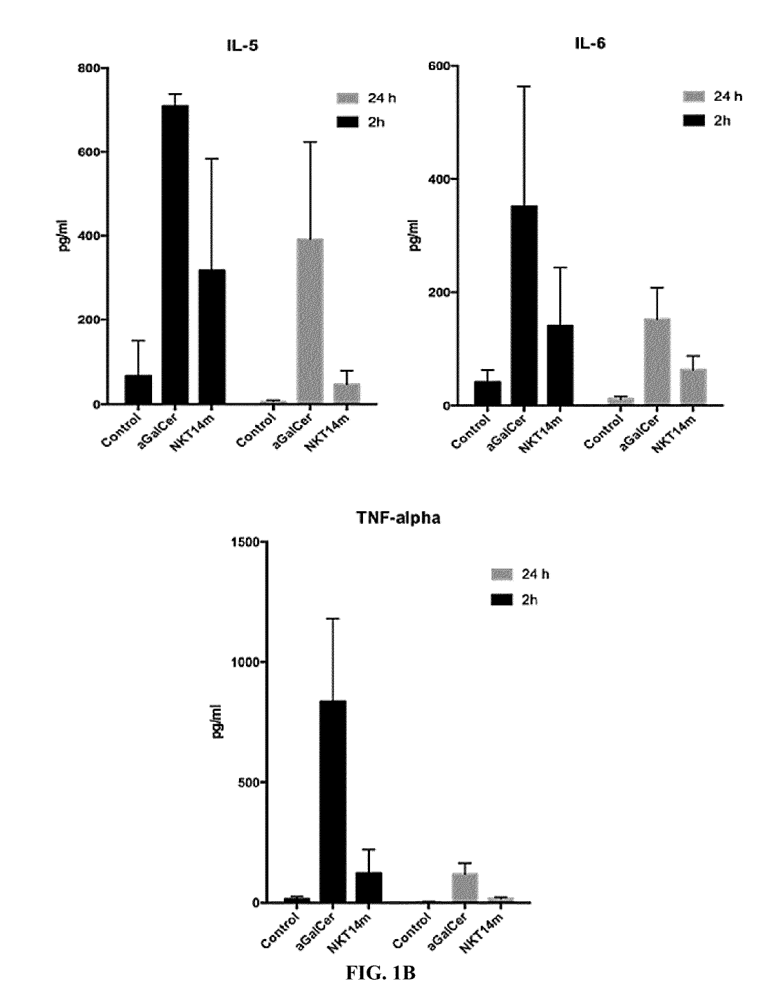

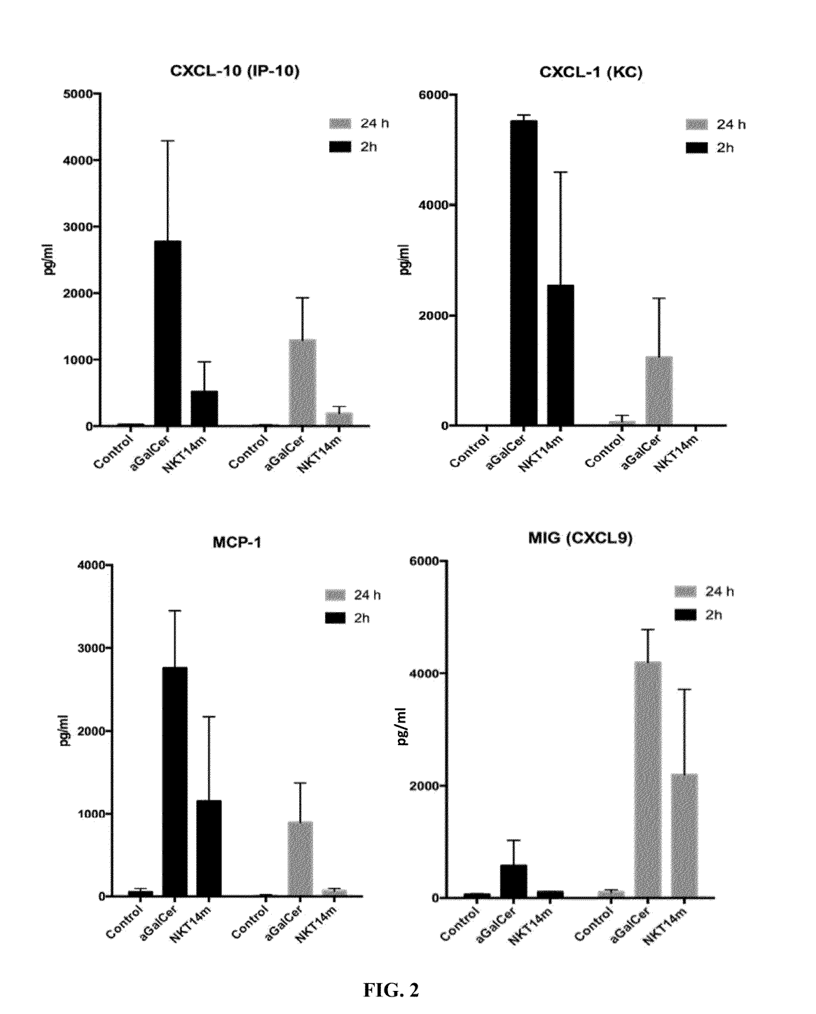

Experiments then were conducted to determine whether NKT14m induces a similar cytokine and chemokine profile as compared to the profile observed in human iNKT cells activated by α-GalCer. As shown in FIGS. 1 and 2, the cytokine and chemokine profile induced by NKT14m was qualitatively similar to that produced by α-GalCer in human iNKT cells.

### EXAMPLE 3

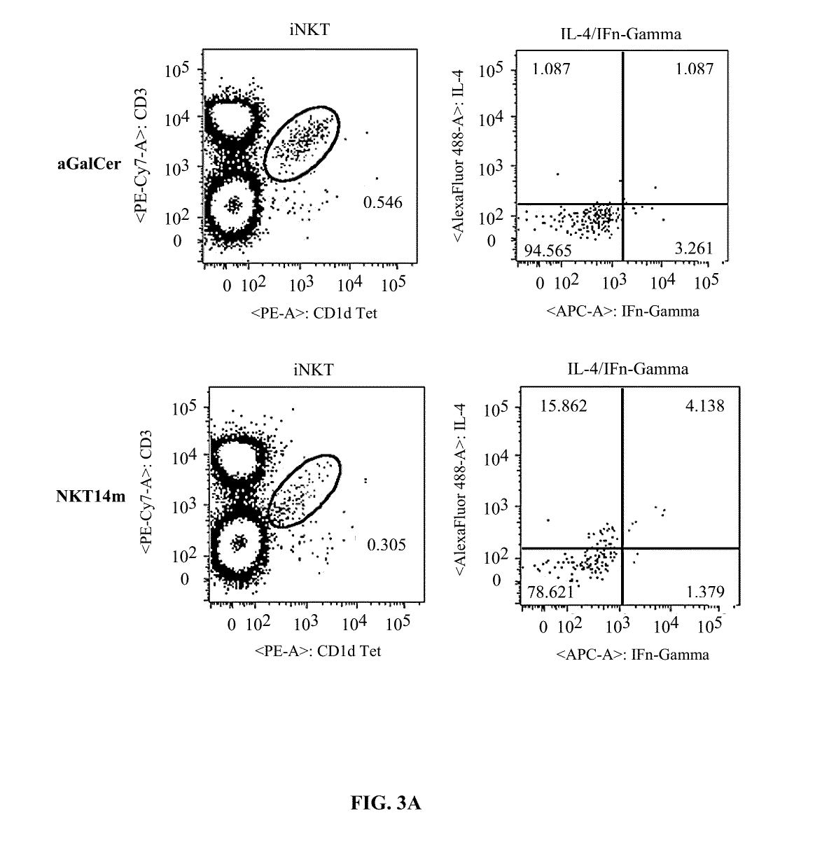

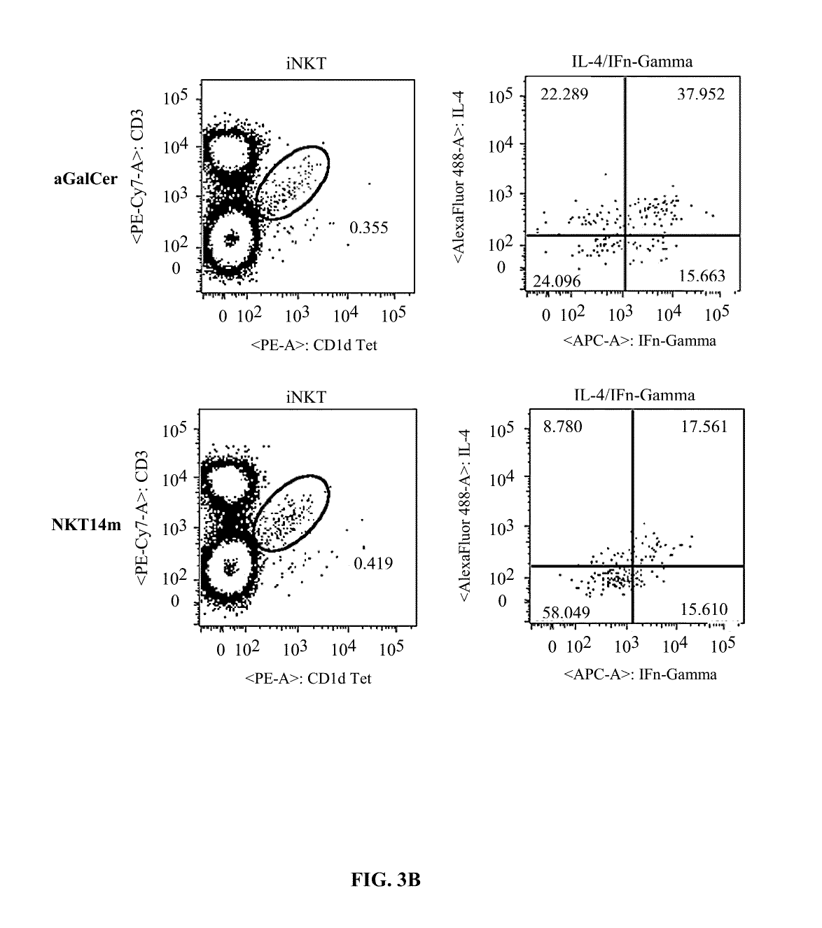

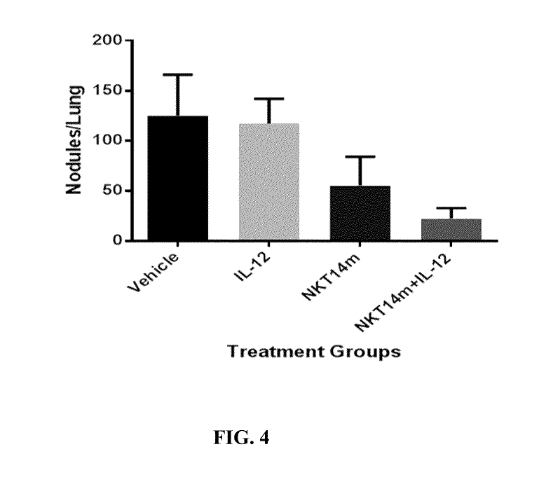

Experiments then were conducted to determine whether NKT14m activation of iNKT cells caused anergy similar to that caused by α-GalCer activation of iNKT cells. The effects on several parameters were measured six weeks post doing with α-GalCer versus NKT14m. Unexpectedly, it was discovered that NKT14m dosing did not cause long lasting anergy similar to that caused by α-GalCer dosing. This suggests that antibody activation of iNKT cells might be a better therapeutic strategy than α-GalCer iNKT cell activation. See FIG. 3.

### EXAMPLE 4

The efficacy of NKT14m alone and in combination with rhIL-12 was assessed in a murine model of cancer.

Study—The study involved 4 groups of C57BL/6 female mice. Each mouse was injected with 400,000 B16F10 tumor cells. Two days after tumor injection, various additional treatments of the animals were started. In particular:

Group 1 mice were treated with vehicle control (5 mice)

Group 2 were treated with IL-12 at a dose of 0.1 ug/mouse IP daily (Days 2-13) (5 mice)

Group 3 mice were treated with NKT-14m at a dose of 100 ug/mouse once on Day 2 (4 mice)

Group 4 mice were treated with NKT-14m at a dose of 100 ug/mouse once on Day 2 plus IL-12 at a dose of 0.1 ug/mouse IP daily (Days 2-13)

All animals were terminated on Day 14. Lungs were removed, fixed in formalin, the number of melanoma nodules were counted per lung and lung were submitted for histology.

The study found that IL-12 alone was ineffective and that there was some reduction in nodules with NKT-14m alone, though not significant in this experiment. However, the combination of IL-12 with NKT-14m produced a significant reduction in the number of metastatic nodules. The IL-12 was administered in a dose that was approximately 5-10-fold less than what is believed to be an optimal dose for IL-12.

### SUMMARY

Overall the results suggest that, in established tumors, NKT14m in combination with rhIL-12 can mediate an anti-tumor effect and permit lower, safer doses of rhIL-12.

We claim:

1. A method for treating a human subject having a cancer or an infection, comprising administering to the human subject an effective amount of
(a) an isolated humanized antibody that selectively binds and activates iNKT cells, and
(b) recombinant human IL-12 (rhIL-12), wherein the isolated humanized antibody and the rhIL-12 are administered in amounts effective to treat the cancer or the infection.

2. The method of claim 1, wherein the rhIL-12 is administered in a dose not exceeding 250 ng/kg per day.

3. The method of claim 1, wherein the rhIL-12 is administered in a dose not exceeding 100 ng/kg per day.

4. The method of claim 1, wherein the rhIL-12 is administered in a dose not exceeding 50 ng/kg per day.

5. The method of claim 1, wherein the rhIL-12 is administered in a single injection between one and three weeks prior to daily consecutive dosing, and then is administered in daily consecutive dosing for at least 5 days and not more than 10 days.

6. The method of claim 1, wherein the subject has cancer and the isolated humanized antibody and the rhIL-12 are administered in amounts effective to treat the cancer.

7. The method of claim 6, wherein the cancer is melanoma, squamous cell carcinoma, basal cell carcinoma, breast cancer, head and neck carcinoma, thyroid carcinoma, soft tissue sarcoma, bone sarcoma, testicular cancer, prostatic cancer, ovarian cancer, bladder cancer, skin cancer, brain cancer, angiosarcoma, hemangiosarcoma, mast cell tumor, primary hepatic cancer, lung cancer, pancreatic cancer, gastrointestinal cancer, renal cell carcinoma, hematopoietic neoplasia, or a metastatic cancer thereof.

8. The method of claim 7, wherein the cancer is melanoma.

9. The method of claim 7, wherein the cancer is metastatic melanoma.

10. The method of claim 1, wherein the isolated humanized antibody and the rhIL-12 are not administered on the same day.

11. The method of claim 1, wherein the subject is not receiving concurrent therapy with α-GalCer.

12. The method of claim 1, wherein the isolated humanized antibody that selectively binds and activates iNKT cells comprises NKTT320.

13. A kit comprising a package containing (a) an isolated humanized antibody that selectively binds and activates iNKT cells, and (b) rhIL-12.

14. The kit of claim 13, wherein the isolated humanized antibody that selectively binds and activates iNKT cells is iNKT320.

15. The kit of claim 14, wherein the isolated humanized antibody that selectively binds and activates iNKT cells and the rhIL-12 are in separate containers within the package.
